(19) United States
(12) Patent Application Publication
(10) Pub. No.: US 2021/0155674 A1
(43) Pub. Date: May 27, 2021

(54) FACTOR IX-TRANSFERRIN FUSION PROTEINS

(71) Applicant: WESTERN UNIVERSITY OF HEALTH SCIENCES, Pomona, CA (US)

(72) Inventors: Jinghua Jeffrey WANG, Arcadia, CA (US); Chen XIE, Chino Hills, CA (US); Zhijun WANG, Chino Hills, CA (US); Yang SU, South Pasadena, CA (US)

(21) Appl. No.: 16/626,010

(22) PCT Filed: Jun. 29, 2018
(86) PCT No.: PCT/US2018/040366

§ 371 Date: Dec. 23, 2019

Related U.S. Application Data
(60) Provisional application No. 62/527,347, filed on Jun. 30, 2017.

(51) Int. Cl. C07K 14/745; A61P 7/00; C07K 14/79

(52) U.S. Cl. CPC C07K 14/745; C07K 2319/00; C07K 14/79; A61P 7/00

(57) ABSTRACT

The invention relates to fusions of coagulation Factor IX (FIX) and transferrin (Tf). These FIX-Tf fusion proteins can be administered orally, and are capable of reaching the systemic circulation by utilizing an endocytosis-dependent mechanism to cross the gut epithelium. Upon delivery of FIX-Tf fusion proteins to the systemic circulation, the fusion proteins are useful for treating bleeding disorders, such as haemophilia B.

### FIELD OF THE INVENTION

The invention relates to blood coagulation Factor IX (FIX) fusion proteins useful for treating bleeding disorders.

### BACKGROUND

Coagulation factor IX (FIX) is a serine protease essential for hemostasis. Deficiency of this protein causes the severe bleeding disorder, hemophilia B, also known as Christmas disease. The standard care for hemophilia B patients is replacement therapy with plasma derived or recombinant FIX. This can be either for episodic treatment or prophylaxis. However, the rapid in vivo clearance of administered FIX creates the need for multiple injections. Thus, improved variants of FIX with an extended half-life and comparable pro-coagulatory properties to wild type FIX have a high potential to reduce the number of injections per bleeding episode. Furthermore, the availability of an oral form of FIX would greatly improve patient compliance and thus therapeutic and prophylaxis outcomes. In that regard, the fusion proteins described below take advantage of Tf-mediated endocytosis as a mechanism to deliver biologically active FIX to the systemic circulation following its oral administration.

### SUMMARY OF THE INVENTION

This disclosure relates to fusions of coagulation Factor IX (FIX) and transferrin proteins, linked together by a peptide linker. These fusion proteins are capable of crossing the gut epithelium via an endocytosis-dependent process. Upon passage through the gut epithelium, the fusion proteins can access the systemic circulatory system to deliver a functional Tf protein useful for treating coagulation disorders associated with insufficient or aberant FIX activity, such as hemophilia B.

### BRIEF DESCRIPTION OF THE FIGURES

FIG. 1A shows gel electrophoresis analysis of the FIX-Linker-Tf-pCDNA 3.1(+) expression construct following endonuclease digestion. Lane 1-1 shows undigested FIX-Tf-pCDNA 3.1(+), Lane 1-2 shows AFLII and Xbal digestion products, and Lane 1-3 shows Xhol and Xbal digestion products.

FIG. 1B shows gel electrophoresis analysis of the FIX-A(EAAAK)₅A-Tf-pCDNA 3.1(+) expression construct following endonuclease digestion. Lane 1-1 shows undigested FIX-A(EAAAK)₅A-Tf-pCDNA 3.1(+), and Lane 1-2 shows AFLII and Xbal digestion products. A(EAAAK)₅A=(SEQ ID NO: 8).

FIG. 1C shows gel electrophoresis analysis of FIX-A(EAAAK)₂A-Tf-pCDNA 3.1(+) and FIX-SVSQTSKLTRAETVFPDVDGS-Tf-pCDNA 3.1(+) expression constructs, in Lanes (1-1 to 1-3), (2-1 to 2-3), following endonuclease digestion. Lane 1-1 shows undigested FIX-A(EAAAK)₂A-Tf-pCDNA 3.1(+); Lane 1-2 shows AFLII and Xbal digestion products; Lane 1-3 shows AFLII, Xhol and Xbal digestion products. Lane 2-1 shows undigested FIX-SVSQTSKLTRAETVFPDVDGS-Tf-pCDNA 3.1(+), Lane 2-2 shows AFLII and Xbal FIX-SVSQTSKLTRAETVFPDVDGS-Tf-pCDNA 3.1(+) digestion products; and 2-3 shows AFLII, Xhol and Xbal FIX-SVSQTSKLTRAETVFPDVDGS-Tf-pCDNA 3.1(+) digestion products. A(EAAAK)₂A=(SEQ ID NO: 7); SVSQTSKLTRAETVFPDVDGS=(SEQ ID NO: 10).

FIG. 1D shows gel electrophoresis analysis of FIX-(GGGGS)₂-Tf-pCDNA 3.1(+), FIX-dithiocyclopeptide-Tf-pCDNA 3.1(+), and FIX-(GGGGS)₂-Tf-pCDNA 3.1(+) expression constructs in Lanes (1-1 to 1-3), (2-1 to 2-3) and (3-1 to 3-3), respectively, following endonuclease digestion. Lane 1-1 shows undigested FIX-(GGGGS)₂-Tf-pCDNA 3.1(+); Lane 1-2 shows AFLII and Xbal digestion products; and Lane 1-3 shows AFLII, Xhol and Xbal digestion products. Lane 2-1 shows undigested FIX-dithiocyclopeptide-Tf-pCDNA 3.1(+), Lane 2-2 shows AFLII and Xbal FIX digestion products; and 2-3 shows AFLII, Xhol and Xbal digestion products. Lane 3-1 shows undigested FIX-(GGGGS)-Tf-pCDNA 3.1(+), Lane 3-2 shows AFLII and Xbal FIX digestion products; and Lane 3-3 shows AFLII, Xhol and Xbal digestion products. (GGGGS)₂=(SEQ ID NO: 5); (GGGGS)₅s=(SEQ ID NO: 6).

FIG. 2A shows a Commassie blue-stained SDS-PAGE analysis of serum-free, conditioned media collected from a single 150 cm² culture dish of HEK293 cells transfected with FIX-Tf-pCDNA 3.1(+) expression systems for each of the following: Negative control (Lane 1); FIX-(GGGGS)₂-Tf (Lane 2); FIX-(GGGGS)₂-Tf (Lane 3); FIX-A(EAAAK)₂A-Tf (Lane 4); FIX-(EAAAK)₅-Tf (Lane 5); FIX-dithiocyclopeptide-Tf (Lane 6); FIX-SVSQTSKLTRAETVFPDVDGS-Tf (Lane 7); and FIX-Tf (Lane 8). One approximately 130 kDa band corresponds to the FIX-Tf fusion protein. (GGGGS)₂=(SEQ ID NO: 5); (GGGGS)₅=(SEQ ID NO: 6); A(EAAAK)₂A=(SEQ ID NO: 7); A(EAAAK)₅A=(SEQ ID NO: 8); SVSQTSKLTRAETVFPDVDGS=(SEQ ID NO: 10).

FIG. 2B shows a Western blot analysis of FIX-Linker-Tf recombinant fusion protein expression by HEK293 cells in culture. Fusion proteins were detected using anti-transferrin antibodies as primary antibodies to probe the blot. The results shown in each lane, 1-8, are based on conditioned media collected from a 150 cm² culture dish of HEK293 cells transfected with FIX-Tf-pCDNA 3.1(+) expression systems for: FIX-(LE)-Tf (Lane 1); FIX-(GGGGS)₅-Tf (Lane 2); FIX-A(EAAAK)₂A-Tf (Lane 3); FIX-A(EAAAK)₅A-Tf (Lane 4); FIX-SVSQTSKLTRAETVFPDVDGS-Tf (Lane 5); FIX-dithiocyclopeptide-Tf (Lane 6); FIX-(GGGGS)₂-Tf (Lane 7); FIX-(LE)-Tf (Lane 8); negative control Lane 9. (GGGGS)₂=(SEQ ID NO: 5); (GGGGS)₅=(SEQ ID NO: 6); A(EAAAK)₂A=(SEQ ID NO: 7); A(EAAAK)₅A=(SEQ ID NO: 8); SVSQTSKLTRAETVFPDVDGS=(SEQ ID NO: 10).

FIG. 2C shows a Western blot analysis of FIX-Linker-Tf recombinant fusion proteins expression by HEK293 cells transfected with FIX-Linker-Tf expression constructs. Fusion proteins were detected in cell culture medium, using anti-FIX antibodies as primary antibodies to probe the blot. The results shown in each lane, 1-8, are based on conditioned media collected from a 150 cm² culture dish of HEK293 cells transfected with FIX-Tf-pCDNA 3.1(+) expression systems for: FIX-(LE)-Tf (Lane 1); FIX-(GGGGS)₅-Tf (Lane 2); FIX-A(EAAAK)₂A-Tf (Lane 3); FIX-A(EAAAK)₅A-Tf (Lane 4); FIX-SVSQTSKLTRAETVFPDVDGS-Tf (Lane 5); FIX-dithiocyclopeptide-Tf (Lane 6); FIX-(GGGGS)₂-Tf (Lane 7); negative control (Lane 8); FIX-(LE)-Tf (Lane 9).

FIG. 3A shows a chromatogram for the size exclusion chromatography (SEC) purification of a FIX-(LE)-Tf fusion protein (SEQ ID NO: 16) from conditioned media. The elution of FIX-(LE)-Tf fusion protein corresponds with the first peak, as indicated in the figure, by collection of eluent in tubes 23-35.

FIG. 3B shows a Coomassie blue stained polyacrylamide gel analysis FIX-(LE)-Tf fusion protein collected in SEC collection tubes. Lane 1 is a FIX-(LE)-Tf fusion protein sample control. Lanes 2-8 show protein eluted in elution collection tubes 8, 12, 23, 25, 30, 32, and 35, respectively, including the presence of band that corresponds with high-purity FIX-(LE)-Tf fusion protein in lanes 4-8.

FIG. 3C shows a Coomassie blue stained polyacrylamide gel analysis of a FIX-(LE)-Tf fusion protein preparation sample before and after SEC purification, in lanes 1 and 2, respectively.

FIG. 4 shows specific apical-to-basolateral transcytosis of BeneFIX® and FIX-(LE)-Tf fusion proteins across Caco-2 cell monolayers. Each data point represents the mean±SEM (n=3).

FIG. 5 shows TfR-binding affinity of various Tf-FIX fusion proteins and BeneFIX® in Caco-2 cells. Each data point represents the mean±SEM (n=3).

FIG. 6A shows the in vivo efficacy of i.v. administered fusion proteins and BeneFIX® in treating acute bleeds in hemophilia B mice (B6.129P2-F9tm1Dws/J mice). Bleeds obtained from WT mice served as controls. Hemophilia B mice were left untreated, or administered either: 50 IU/kg of rFIX-Tf/G₂; 20 IU/kg of rFIX-Tf/G₂; 20 IU/kg of rFIX-Tf/SVSQ; or 20 IU/kg of BeneFIX®. The blood loss was determined by quantifying the amount of hemoglobin for each group. *p<0.05, ***p<0.001, ns=not significant (p>0.05).

FIG. 6B shows the in vivo efficacy of orally administered fusion proteins and BeneFIX® in treating acute bleeds in hemophilia B mice (B6.129P2-F9tm1Dws/J mice). Bleeds obtained from WT mice served as controls. Hemophilia B mice were left untreated, or administered either: 200 IU/kg of rFIX-Tf/G₂; 200 IU/kg of rFIX-Tf/SVSQ; or 200 IU/kg of BeneFIX®. The blood loss was determined by quantifying the amount of hemoglobin for each group. *p<0.05, ***p<0.001, ns=not significant (p>0.05).

FIG. 7 shows pharmacokinetic profiles, over the course of 72 hours, in wild type mice following i.v. administrations of 50 IU/kg rFIX-Tf/G₂, rFIX-Tf/SVSQ or BeneFIX®. Plasma levels were determined by a FIX specific ELISA kit. The results shown represent the FIX levels of the plasma of three animals per time point for each group.

FIG. 8 shows pharmacokinetic profiles, over the course of 72 hours, in wild type mice following oral administrations of 200 IU/kg rFIX-Tf/G₂, rFIX-Tf/SVSQ or BeneFIX®. Plasma levels were determined by a FIX specific ELISA kit. The results shown represent the FIX levels of the plasma of three animals per time point for each group.

FIG. 9 shows a schematic diagram of steps taken to identify promising rTIX-Tf fusion proteins.

### DETAILED DESCRIPTION

Fusions of coagulation Factor IX (FIX) and transferrin are described herein. Accordingly, a fusion protein according to the invention includes a FIX-T component and a Tf component. More particularly, a fusion protein according to the invention includes a peptide linker, which serves to link the FIX and Tf components. The FIX and Tf components are linked, such that the amino-to-carboxy order of the components of a fusion protein according to the invention is FIX component>Peptide Linker>Tf component. Fusion proteins according to the invention are capable of crossing the gut epithelium via an endocytosis-dependent process mediated by binding of the Tf component to a Tf receptor (TfR). After crossing the gut epithelium, the fusion proteins can reach the systemic circulatory system, whereupon, the Tf component is useful for treating coagulation disorders associated with insufficient or aberant FIX activity. Therefore, fusion proteins according to the invention can be utilized to deliver rFIX for use in treating coagulation disorders associated with insufficient or aberrant FIX activity.

A “FIX component” of a fusion protein of the invention is a protein domain that retains the biological functions of FIX, such as functioning as a clotting factor. Human FIX is a 415 amino acid long polypeptide with a molecular weight of approximately 57 kDa, and is synthesized in the liver and secreted as a zymogen (an inactive pro-enzyme) into the bloodstream. A FIX component according to the invention can have the wild-type amino acid sequence of a FIX protein (e.g., a human FIX protein), or a variant of the wild-type FIX. A variant FIX protein may have one or more amino acid deletions, insertions, nonconserved or conserved substitutions, or combinations thereof, of the amino acid sequence of a native mammalian FIX, as long as they result in no substantial alterations of the active site(s) or domain(s) that mediate its function as a clotting factor. Examples of FIX components of fusion proteins of the invention include the amino acid sequences associated with: Entry EC 3.4.21.22 in SIB's Bioinformatics Resource Portal, ExPASy; a protein encoded by the human gene located on the X chromosome (Xq27.1-q27.2); the amino acid sequence identified by SEQ ID NO: 2, or an amino acid sequence that is at least: 90%, 91%, 92%. 93%, 94%, 95%, 96%, 97%, 98%, 99%, or 100% homologous with the sequence of SEQ ID NO: 2.

Serum transferrin (Tf) is a glycoprotein of approximately 80 kDa in size that binds and transports non-heme iron. An iron-bound Tf protein binds a transferrin receptor (TfR) on the surface of a cell, such as an erythroid precursor or an epithelial cell, and is subsequently transported into a cell vesicle by receptor-mediated endocytosis. Following the release of iron ions by the Tf protein, Tf and TfR are then transported through the endocytic cycle back to the cell surface, ready for another round of iron uptake. TfR-mediated endocytosis can also be used for increasing epithelial absorption of Tf-linked drugs.

A “Tf component” of a fusion protein according to the invention is a protein domain that binds a TfR. A Tf component may have the wild-type amino acid sequence of a Tf protein (e.g., a human Tf protein), or a variant of the wild-type Tf. A variant Tf protein may have one or more amino acid deletions, insertions, nonconserved or conserved substitutions, or combinations thereof, of the amino acid sequence of a native mammalian Tf protein, as long as they result in no substantial alterations of the active site or domain responsible for the biological activity of Tf. The activity of a Tf domain may be determined using any of the methods known in the art. For example, the activity of a Tf domain may be determined by measuring its ability to bind a TfR. Examples of Tf components of fusion proteins of the invention have an amino acid sequence of human Tf, as defined by SEQ ID NO: 4, or an amino acid sequence that is at least: 90%, 91%, 92%. 93%, 94%, 95%, 96%, 97%, 98%, 99%, or 100% homologous with the sequence of SEQ ID NO: 4.

The linker peptide of a fusion protein according to the invention may be noncleavable or cleavable by proteases such as thrombin, factor Xa, and factor Xia, or a protease typically present in damaged tissue. Cleavable peptide linkers may, for example, include the peptides, dithiocyclopeptide (SEQ ID NO: 9) or SVSQTSKLTRAETVFPDVDGS (SEQ ID NO: 10). Exemplary fusion proteins of the invention that include either a dithiocyclopeptide cleavable linker, or cleavable linker with the sequence SVSQTSKLTRAETVFPDVDGS contain the amino acid sequences identified by SEQ ID NOS: 24 and 14, respectively. Noncleavable peptide linkers may, for example, include the peptides: (GGGGS)₂ (SEQ ID NO: 5); (GGGGS)₅ (SEQ ID NO: 6); A(EAAAK)₂A (SEQ ID NO: 7); or A(EAAAK)₅A (SEQ ID NO: 8). Exemplary fusion proteins of the invention that include any of the noncleavable linkers (GGGGS)₂, (GGGGS)₅, A(EAAAK)₂A, or A(EAAAK)₅A contain the amino acid sequences identified by SEQ ID NOS: 12, 22, 20, and 25, respectively. Fusion proteins of the invention may also include a dipeptide sequence, which follows the carboxy end of the linker, and is positioned at the amino end of the transferrin component of the fusion protein. For example, the dipeptide sequence leucine-glutamic acid (“LE”), may be positioned between the linker and transferrin components of a fusion protein of the invention.

As indicated above, the invention also relates to recombinant expression systems for producing fusion proteins of the invention. Recombinant expression systems generally include polynucleotide sequences, which encode fusion proteins of the invention are also disclosed. For example, polynucleotide sequences, encoding FIX-linker-Tf fusion proteins of the invention with the linkers: LE, (GGGGS)₂; (GGGGS)₅; A(EAAAK)₂;A; A(EAAAK)₅A; dithiocyclopeptide (SEQ ID NO: 9) or SVSQTSKLTRAETVFPDVDGS, respectively, are associated with: SEQ ID NO: 16; SEQ ID NO: 12; SEQ ID NO: 22; SEQ ID NO: 20; SEQ ID NO: 18; SEQ ID NO: 24; and SEQ ID NO: 14, respectively.

Fusion proteins according to the invention can be produced using recombinant expression systems. Thus, a polynucleotide, that encodes a fusion protein of the invention can be inserted into an expression vector for the production of recombinant fusion proteins. Expression vectors may be constructed to encompass a signal sequence for membrane targeting or secretion or a leader sequence. Alternatively, a polynucleotide, encoding a fusion protein of the invention, may include its own signal sequence, and not rely on a signal or leader sequence of the expression vector. Vectors of the invention may also include regulatory sequences, such as a promoter, an operator, an initiation codon, a termination codon, a polyadenylation signal, an enhancer In addition, the expression vector may contain a selection marker for selecting host cells transformed with the expression vector and a replication origin in case of a replicable expression vector. The vector can replicate by itself or can be incorporated into a chromosome of the host cell. A recombinant expression vector according to the invention may be constructed by inserting a polynucleotide, encoding a fusion protein of the invention, into a pcDNA3.1(+) vector, for example.

Polynucleotides encoding the fusion proteins of the invention may have various modifications made in the encoding region within the extent that they do not change the amino acid sequence of a fusion protein, due to codon degeneracy or in consideration of the codons preferred by the organism in which they are to be expressed, and various modifications or alterations may be introduced in regions other than the coding region so long as they have no influence on the expression of the gene.

The invention also includes a host cell, transformed with a recombinant expression vector according to the invention. Examples of host cells useful in the invention include Chinese hamster ovary (CHO) cells, human embryonic kidney cells (HEK293), baby hamster kidney cells (BHK-21), and the human hepatic carcinoma cell line (HepG2). A recombinant expression vector of the present invention can be introduced into host cells using conventional techniques known in the art, including electroporation, protoplast fusion, viral transfection, cationic lipid transfection, DEAE-Dextran transfection, cationic polymers, calcium phosphate (CaPO₄) co-precipitation, and calcium chloride (CaCl₂) precipitation.

A fusion protein of the invention, which accumulates in the medium of transformed, fusion protein-secreting cells of the above types, can be concentrated and purified by a variety of biochemical and chromatographic methods, including methods utilizing differences in size, charge, hydrophobicity, solubility, and specific affinity, between the desired fusion protein and other substances in the cell cultivation medium.

The invention also relates to methods of treating a bleeding disorder in a subject in need thereof, by administering a therapeutically effective amount of a fusion protein according to the invention to the subject. A therapeutically effective amount of a fusion protein improves the ability of the subject's blood to clot. Bleeding disorders that are effectively treated by administering a fusion protein of the invention include, but are not limited to disorders caused by defects in the function or expression of FIX. For example, a bleeding disorder effectively treated by administration of a fusion protein of the invention is hemophilia B, also known as Haemophilia B or Christmas Disease, a disorder that can be caused by genetic defects in the expression of functional FIX.

As a fusion protein according to the invention can be used to treat bleeding disorders, the invention also provides pharmaceutical compositions for fusion proteins according to the invention, as well as methods of delivering those compositions. More particularly, a pharmaceutical composition according to the invention can be administered orally, topically, parenterally, by inhalation spray, vaginally, rectally, or by intracranial injection. Indeed, a pharmaceutical composition according to the invention may be administered by any convenient route, such as, for example by infusion or bolus injection, by absorption through epithelial or mucocutaneous linings (e.g., oral mucosa, rectal and intestinal mucosa, etc.)

A pharmaceutical composition according to the invention can be formulated with at least one, or any combination of, a pharmaceutically acceptable carrier, diluent, salt, buffer, or excipient appropriate for oral, topical, parenteral (for example, subcutaneous injections, intravenous, intramuscular, intracisternal injection, or infusion techniques), inhalation, vaginal, rectal, or intracranial administration. The foregoing compositions are essentially free of pyrogens, as well as other impurities that could be harmful to the recipient. In general, the term “pharmaceutically acceptable” indicates approval by a regulatory agency of a national government, or inclusion in the U.S. Pharmacopeia, or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A pharmaceutical composition according to the invention, if desired, can also contain minor amounts of wetting or emulsifying agents, or pH buffering agents. Therefore, such compositions can take the form of solutions, suspensions, emulsion, tablets, pills, capsules, powders, sustained-release formulations and the like.

An oral dosage form of a pharmaceutical composition according to the invention of can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Oral dosage forms can also be obtained by direct compression of active dry powders containing a fusion protein according to the invention, mixed with selected excipients, such as cellulose derivatives, metacrylates, chitosan, carboxymethylstarch (CMS), or mixtures thereof to form a tablet. Alternatively, an oral dosage form according to the invention may be prepared as a capsule containing multiparticulates, powders, or both, of compression of active dry powders containing a fusion protein according to the invention, mixed with selected excipients, such as cellulose derivatives, metacrylates, chitosan, or CMS.

The invention also relates to kits which comprise a composition of the invention packaged in a manner which facilitates its use for administration to subjects. Such a kit includes a composition described herein (e.g., a composition comprising recombinant fusion protein of the invention), packaged in a container such as a sealed bottle or vessel, with a label affixed to the container or included in the package that describes use of the compound or composition in practicing the method. The kit may contain a first container having a pharmaceutical composition comprising a recombinant fusion protein of the invention and a second container having a physiologically acceptable reconstitution solution for the composition in the first container. The composition may be packaged in a unit dosage form. The kit may further include a device suitable for administering the composition according to a specific route of administration. The kit may also contain a label that describes use of the therapeutic protein or peptide composition.

### EXAMPLES

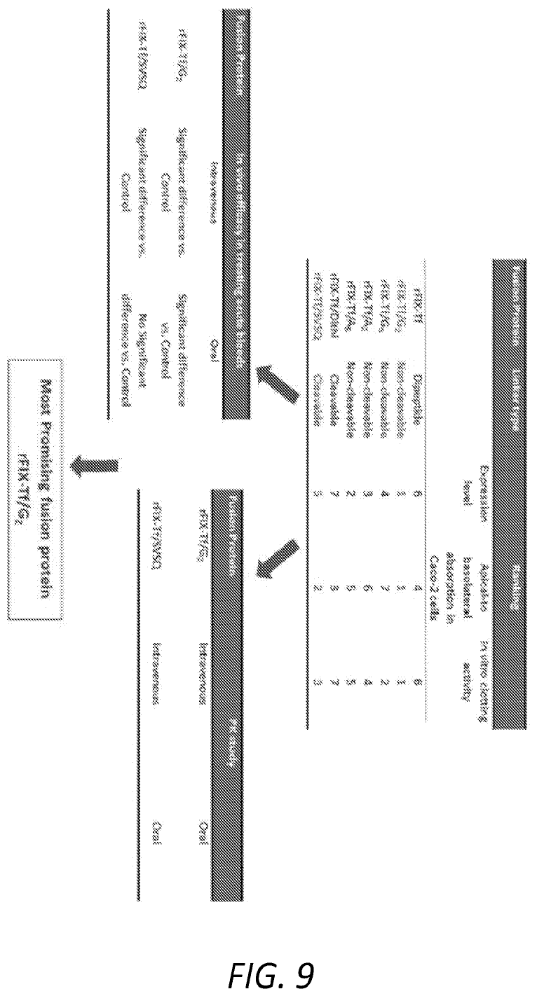

The following examples chronicle the construction and exemplification of recombinant FIX-Tf fusion proteins, including variants of FIX with an extended half-lives and comparable pro-coagulatory properties to wild type FIX. FIG. 9 contains a schematic of the experimental approach used to assess the expression, functionality and efficacy of the FIX-Tf fusion protein variants described herein.

Example 1. Cloning of recombinant FIX fusion proteins. FIX wild-type cDNA was prepared for genetic fusion to transferrin by introduction of a restriction site Xhol replacing the natural FIX stop codon. The cDNA encoding the entire Human FIX, excluding its TAA stop codon, in-frame with the linker A(EAAAK)₅A, was synthesized by Gencript USA Inc. (Piscataway, N.J., USA). The restriction recognition sites of AFLII and Xhol were also added. The coding area of human FIX was amplified by PCR with primers identified by SEQ ID NO: 27 and SEQ ID NO: 28, which incorporated the Xhol and AFLII restriction enzyme sites. The primer sequences are listed in Table 1.

TABLE 1

| | Primer | Restriction | | |
|---|---|---|---|---|
| Gene | Sequence | Site | bp | Note |
| Human | Tf-f: ACCGCTC | XhoI | 2083 | Without |
| Tf | GAGGTCCCTGATA | (ACCGC | | signal |
| | AAACTGTGAGAT | TCGAG) | | sequence |
| | (SEQ ID NO: 25) | XbaI | | |
| | Tf-r: GTAGTCTA | (GTAGT | | |
| | GATTAAGGTCTACG | CTAGA) | | |
| | GAAAGTGCA | | | |
| | (SEQ ID NO: 26) | | | |
| Human | F9-f: CTTAAGAC | AfIII | 1424 | With |
| FIX | CACTTTCACAATCT | (CTTAAG) | | signal |
| | GCTAG | XhoI | | sequence |
| | (SEQ ID NO: 27) | (CTCGAG) | | |
| | F9-r: CTCGAGAG | | | |
| | TGAGCTTTGTTTTT | | | |
| | TTCCT | | | |
| | (SEQ ID NO: 28) | | | |

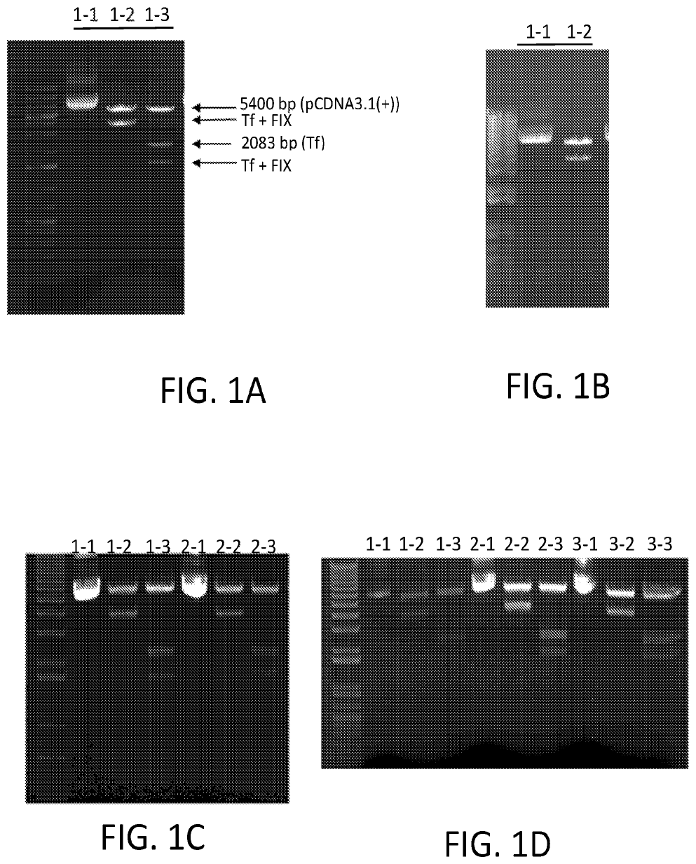

The coding sequence of transferrin, without signal sequences, were amplified by PCR from the plasmid TFR27A (American Type Culture Collection, Manassas, Va., USA) using forward and reverse primers identified by SEQ ID NO: 25 and SEQ ID NO: 26, respectively. The Tf-specific primers incorporated Xhol and Xbal restriction enzyme sites. The Tf fragment was cloned into pCDNA 3.1(+). Subsequently, the Tf fragment was excised from the construct by digestion with Xhol and Xbal, and ligated into an Xhol and Xbal digested FIX-pCDNA 3.1(+). A dipeptide Leu-Glu (LE) was introduced between the FIX and Tf as a consequence of introducing the Xhol restriction site. FIX sequences with the various linker sequences, including restriction recognition sites of Xhol and AFLII, were synthesized by Genscript USA Inc. (Piscataway, N.J., USA), respectively. Table 2 contains the nucleotide sequences of the linkers. Synthesized FIX DNA with linker insert was then cloned into the pcDNA3.1(+) vector. FIX-linker fragments were digested with Xhol and AFLII, and ligated into an Xhol/AFLII digested Tf-pCDNA 3.1(+) plasmids. Correct placement of the FIX-linker-Tf expression construct components were confirmed by enzymatic digestion with AfLll and Xbal. Following digestion, two separated bands corresponding to the 5400 bp for pCDNA 3.1(+) vector and nearly 3500 bp for the fused FIX-Tf fragment could be identified by gel electrophoresis. The competed constructs were also confirmed by triple enzyme digestion to yield three separate bands in 5400 bp for pCDNA 3.1(+) vector, 1424 bp for FIX fragment and 2083 bp for Tf fragment are consistent with expected fragment size. The assembled sequencing data was compared to published Tf and FIX sequences for verification. Both FIX and Tf were correctly fused in frame without mutations. The various FIX-linker-Tf-pCDNA 3.1(+) expression constructs were confirmed using the same method. See FIG. 1(A-D).

TABLE 2

| FIX-linker-Tf | Linker | |
|---|---|---|
| fusion | Type | Linker AA Sequence |
| rFIX-Tf | Dipeptide | LE |
| (SEQ ID NO: 16) | | |
| rFIX-Tf/G₂ | Non- | (GGGGS)₂ |
| (SEQ ID NO: 12) | cleavable | (SEQ ID NO: 5) + LE |
| rFIX-Tf/G₅ | Non- | (GGGGS)₅ |
| (SEQ ID NO: 22) | cleavable | (SEQ ID NO: 6) + LE |
| rFIX-Tf/A₂ | Non- | A(EAAAK)₂A |
| (SEQ ID NO: 20) | cleavable | (SEQ ID NO: 7) + LE |
| rFIX-Tf/A₅ | Non- | A(EAAAK)₅A |
| (SEQ ID NO: 18) | cleavable | (SEQ ID NO: 8) + LE |
| rFIX-Tf/Dithi | Cleavable | Dithiocyclopeptide |
| (SEQ ID NO: 24) | | (SEQ ID NO: 9) + LE |
| rFIX-Tf/SVSQ | Cleavable | SVSQTSKLTRAET |
| (SEQ ID NO: 14) | | (SEQ ID NO: 10) + LE |

Example 2. Expression and characterization of fusion proteins. HEK293 cells were seeded into 150 cm² cell culture dishes (BD Biosciences, Franklin Lakes, N.J., USA) the day before transfection with plasmids containing the FIX-Tf expression constructs described in Example 1. The cell cultures were 80-90% confluence at the time of transfection. Transfection was performed, using linear polyethylenimine (Polysciences, Warrington, Pa., USA). Following transfection, cells were cultured in conditioned, serum-free CD293 medium supplemented with 1% pen/strep, 10 μg/ml vitamin K1 and 4 mM L-glutamine. The conditioned media containing the fusion protein was collected twice every 3 days and centrifuged at 4000 g for 25 min at 4° C. The supernatant was further concentrated by tangential flow filtration with a molecular mass cut off of 30 kD (TFF; Millipore, Billerica, Mass., USA) to a final volume of 20 ml, and stored at −80° C. until analysis for expression was performed.

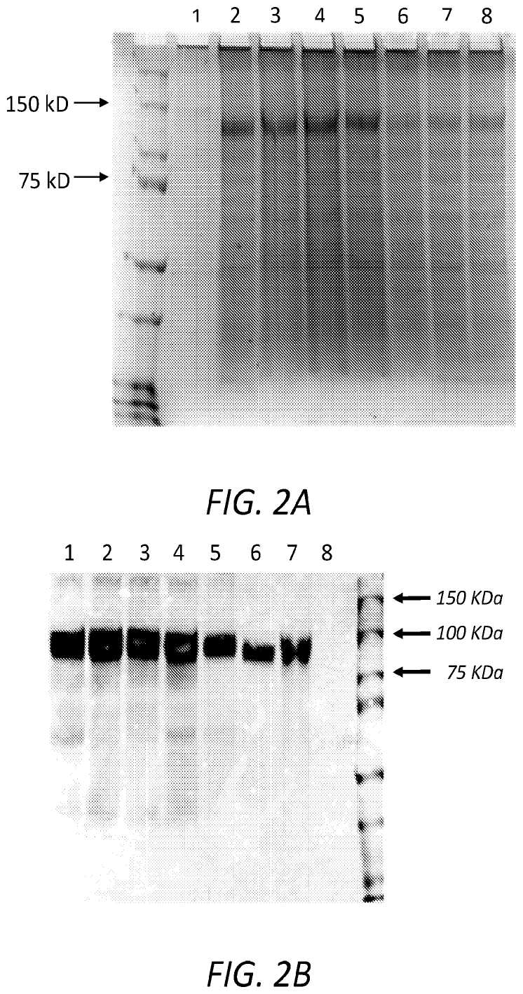

To perform FIX-Tf expression analysis, the concentrated media samples were prepared for SDS-PAGE analysis by removing 60 μl of 20 mL of the concentrated, conditioned media, and added to non-reduced sample buffer, and then boiled for 5 min at 95° C. SDS-PAGE analysis of the serum-free, conditioned media, performed using 8-16% gradient gels demonstrated that one major band with molecular weight corresponding to the fusion protein (130 kDa) was indicative of secretion of expressed FIX-Tf fusion proteins into media. See FIG. 2A. No other 130 kDa proteins were detected in concentrated supernants from cells that were transfected with pCDNA 3.1(+) as the negative control. Expression of the FIX-Tf fusion proteins were measured using a human FIX ELISA kit from AssayPro (Charles, Mo., USA) (“the FIX ELISA assay”). See Table 3.

TABLE 3

| | Expression of FIX:Ag in |
|---|---|
| Sample Designation | one 150 cm² dish (IU) |
| rFIX-Tf | 1.76 |
| rFIX-Tf/G₂ | 5.81 |
| rFIX-Tf/G₅ | 4.04 |
| rFIX-Tf/A₂ | 4.50 |
| rFIX-Tf/A₅ | 4.78 |
| rFIX-Tf/Dithi | 1.04 |
| rFIX-Tf/SVSQ | 1.82 |

To confirm identity of the fusion proteins, the conditioned media for each fusion protein was analyzed using Western blot and probed with both anti-FIX and anti-Tf antibodies. Antibody against human FIX (ab124815, Abcam, Cambridge, Mass., USA) and antibody against human Tf (HPA005692, Sigma-Aldrich, St. Louis, Mo., USA) were used as the primary antibodies. Horseradish peroxidase-conjugated anti-rabbit IgG antibody (#7074, Cell Signaling, Danvers, Mass., USA) was used as the secondary antibody. The peroxidase activity was detected by maximum sensitivity chemiluminescence (#34095, Thermo Scientific, Waltham, Mass., USA) for visualization. See FIG. 2(B-C) for FIX and Tf Western blots, respectively. The data revealed that both antibodies detected the fusion proteins with about 130 kDa molecular weight (FIG. 2b). The expression level of fusion proteins with non-cleavable linkers was much higher than that of fusion proteins with cleavable linkers and the expression level for FIX-Tf/G₂ was the highest among them. See FIGS. 2A-2D.

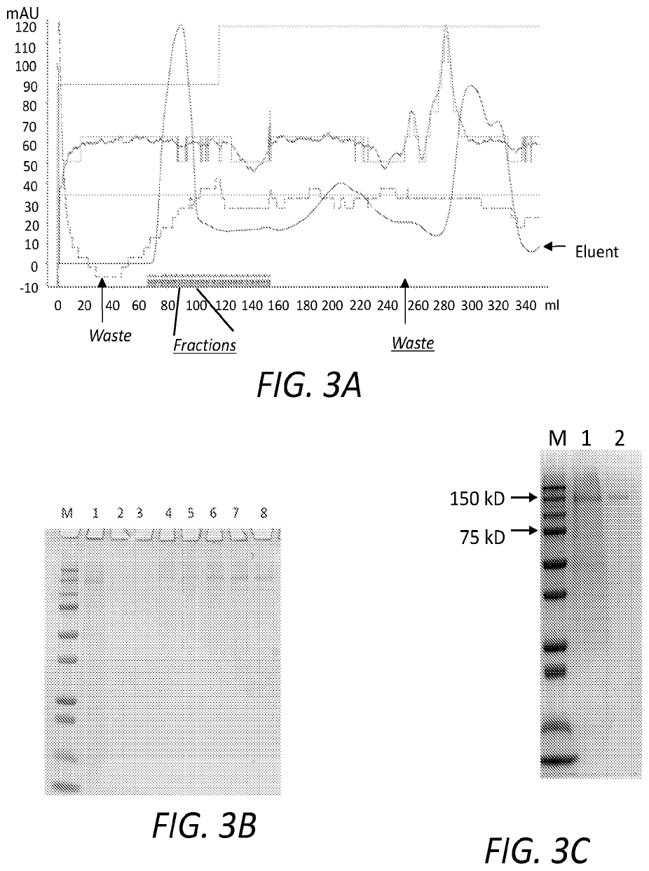

Example 3. Purification of recombinant FIX-(LE)-Tf fusion protein by size exclusion chromatography. Size Exclusion Chromatography (SEC) was performed on a Hiprep 26/60 Sephacryl S-200 HR column (60 cm×26 mm) connected to an AKTA Purifier UPC 100 system (GE Healthcare, Wauwatosa, Wis., USA) and eluted with 30 mM Tris, 100 mM NaCl, pH 6.8 at 1.0 ml/min flow rate. The detection was made at 280 nm. One ml fractions were collected. The protein eluted in fractions 23-35 was collected, and a total of 13 ml of collected fractions were concentrated to a volume of 1.3 ml, using Amicon Ultra-15 centrifuge filter units with a molecular mass cutoff of 50 kDa (Milipore, Billerica, Mass., USA). See FIG. 3(A-C). About 90% of the FIX-(LE)-Tf fusion proteins in the conditioned media was lost during further processing steps after purification.

Example 4. Assessment of apical-to-basolateral transcytosis of BeneFIX® and FIX-Tf fusion proteins across the Caco-2 cell monolayer, an in vitro model for gastrointestinal absorption. Caco-2 cells were grown on 0.4 μm pore size polycarbonate filters in Transwells (Costar, Cambridge, Mass., USA). The transport studies were conducted on 2-week-old Caco-2 monolayers, 6 or 7 days after they have exhibited signs of tight junction development, and exhibited TEER levels of approximately 500 Ω·cm. Monolayers were washed once with DMEM containing 0.1% BSA and incubated at 37° C. for 45 min to deplete endogenous Tf. Media was subsequently replaced and the monolayers treated with BeneFIX® and various fusion protein preparations in the apical compartment (100 mlU/ml). Nonspecific transport was measured in parallel by the inclusion of 100-fold molar excess of Tf. At two, four, and six hours post-dosing, 500 μl samples were collected from the basolateral compartment and replenished with an equal volume of fresh DMEM. The extent of TfR-mediated transcytosis was determined by subtracting nonspecific transport (with 100×excess Tf) from total transport (without Tf). The integrity of the cell monolayer was monitored during the experiment by measuring TEER.

Transported proteins in the basolateral media were detected on the basis of FIX antigen measured using a human FIX ELISA kit from AssayPro (Charles, Mo., USA) (“the FIX ELISA assay”) performed according to the manufacturer's instructions. More specifically, a monoclonal antibody specific for FIX was pre-coated on a 96-well microplate followed by 2-h incubation with samples and a range of dilutions of FIX standards. The samples were sandwiched by the immobilized antibody and biotinylated polyclonal antibody specific for FIX, which was recognized by horseradish peroxidase conjugate. A peroxidase enzyme substrate was added for detection and absorbance was read at 450 nm. Concentrations of test samples were calculated using human FIX standard as a reference. This assay was used to measure human FIX antigen (FIX:Ag) in cell culture supernatant and plasma and did not have cross-reactivity with mouse FIX.

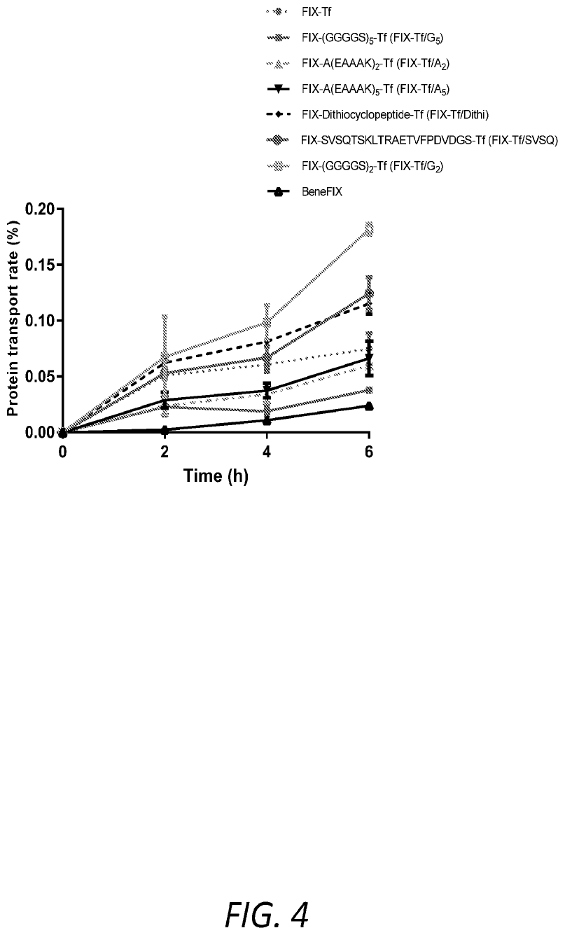

As shown in FIG. 4, the FIX-Tf fusion proteins with non-cleavable linker named FIX-(GGGGS)2-Tf (FIX-Tf/G2) exhibited highest transport rate across Caco-2 cell monolayers at 2 h, 4 h and 6 h, respectively. The amount of transported FIX-Tf/G2 was 7.8-fold higher than BeneFIX® after 6 h incubation (0.187% of protein transport rate for FIX-Tf/G2 and 0.024% of protein transport rate for BeneFIX®). In addition, the transport rates for two FIX fusion proteins with cleavable linker were also significantly higher than the BeneFIX® at 6 h, with FIX-SVSQTSKLTRAETVFPDVDGS-Tf (FIX-Tf/SVSQ) transported 5.8-fold higher than BeneFIX® and FIX-dithiocyclopeptide-Tf (FIX-Tf/Dithi) transported 4.4-fold higher than BeneFIX®. The TEER of Caco-2 cells for cell integrity was not affected within 6 h treatment. Data were analyzed using GraphPad PRISM version 6 (La Jolla, Calif., USA) and presented as mean±SEM. P<0.05 was considered statistically significant.

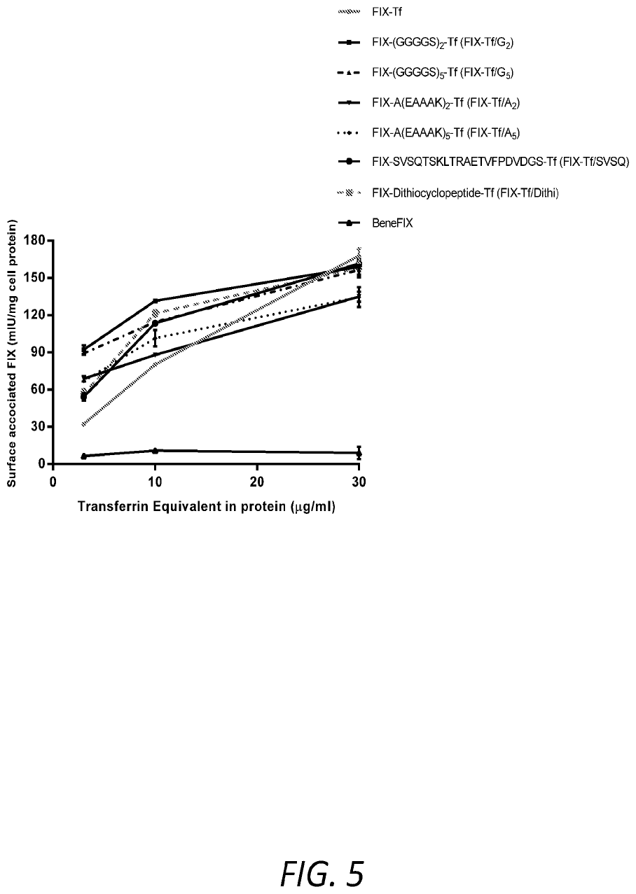

Example 5. TfR binding assay. As shown in FIG. 5, the FIX-Tf fusion proteins were capable of binding transferrin receptors expressed by cultured Caco-2 cells. Therefore, the fusion proteins maintained specific binding ability to TfR. The assay also confirmed that BeneFIX® has no TfR binding activity. To perform the TfR binding assay, Caco-2 cells were seeded in 12-well cluster plates and cultured for 2 weeks until fully differentiated. Caco-2 monolayer was washed with cold PBS three times, and then incubated in serum-free DMEM supplemented with 0.1% bovine serum albumin (BSA) at 37° C. for 30 min to remove any endogenous Tf. A mixture of 3 μg/ml Tf with 3 μg/ml, 10 μg/ml or 30 μg/ml of BeneFIX® and the FIX-Tf fusion proteins in serum-free DMEM supplemented with 0.1% BSA were added to different wells. After 30 min of incubation at 4° C., the medium was removed, and the cell monolayer was washed with cold PBS three times. The cells were then be dissolved in 1 M NaOH and the transferrin in the cell lysates were counted by human FIX ELISA kit from AssayPro (Charles, Mo., USA). The total cellular protein in the cell lysate was measured and used to normalize the data to per mg cell protein. Data were analyzed using GraphPad PRISM version 6 (La Jolla, Calif., USA) and presented as mean±SEM. P<0.05 was considered statistically significant.

Example 6. Determination of in vitro clotting activities of the FIX-Tf fusion proteins. Clotting activities were determined for FIX and FIX-Tf fusion proteins using a one-stage clotting assay based on an activated thromboplastin (aPTT) reagent. Clotting activity correlated to the amount of FIX-Tf fusion proteins in concentrated conditioned media, collected as described in Example 2, and quantitated by comparing the optical density of the CM at 280 and 320 nm as a measure of protein content. Among the different rFIX-Tf preparations described in these Examples, the clotting activity of rFIX-Tf with non-cleavable (GGGGS)₂ is higher than the other fusion proteins described herein. More specifically, in descending order from highest clotting activity, the fusion proteins were ranked as follows, according to data presented in Table 4: rFIX-Tf/G2>rFIX-Tf/G5>rFIX-Tf/SVSQ>rFIX-Tf/A2>rFIX-Tf/A5>rFIX-Tf>rFIX-Tf/Dithi). FIX-deficient human plasma were obtained from Aniara Diagnostica (West Chester, Ohio, USA) and aPTT reagent Pathromtin SL were obtained from Siemens Healthcare Diagnostics (Los Angeles, Calif., USA). For sample measurement, the FIX-deficient plasma was supplemented with BeneFIX® or various FIX-Tf fusion proteins to final concentrations of 6.25-100% FIX. Samples were evaluated against a standard curve prepared with FIX standards. Results are presented in Table 4.

TABLE 4

| Sample Designation | FIX Clotting Activity/OD280-320 (IU/OD) |
|---|---|
| rFIX-Tf | 0.075 |
| rFIX-Tf/G₂ | 0.199 |
| rFIX-Tf/G₅ | 0.185 |
| rFIX-Tf/A₂ | 0.096 |
| rFIX-Tf/A₅ | 0.094 |
| rFIX-Tf/Dithi | 0.073 |
| rFIX-Tf/SVSQ | 0.118 |

Example 7. Selection of fusion rFIX-Tf proteins for in vivo efficacy. For the assessment of in vivo efficacy of rFIX-Tf fusion proteins, male hemophilia B mice (B6.129P2-F9tm1Dws/J strain) were used, and wild type mice were used for comparison. B6.129P2-F9tm1Dws/J mice were bred according to the protocol approved by the Institutional Animal Care and Utilization Committee (IACUC) at Western University of Health Sciences. The animal room was at a controlled temperature of 21-23° C. and a 12 h light-dark cycle. The heterozygous female mice were firstly crossed with wild-type male mice to generate heterozygous female or hemizygous male. Then the strain was maintained through homozygote female crossed with hemizygous male. Wild-type female mice were crossed with wild-type male mice to generate wild type mice. When the mice were 30 days old, they were ear tagged and a 2 mm² piece of mouse ear was removed for genotyping. Sequence information for the PCR primers used for used for genotyping is shown in Table 5. The GoTaq® Green master mix (M7122, Promega, Madison, Wis., USA) was used for performing the PCR reactions. The PCR products were analyzed using agarose gel electrophoresis (1.5% in TBE buffer). The gel was visualized under UV light and sized compared with 1 kb plus DNA ladder. For mutant mice (−/− or −/Y), the band at about 550 bp was expected. For Heterozygous mice (+/−), the bands at about 320 bp and 550 bp were expected. For Wild type (+/+ or +/Y), the band at about 320 bp was expected.

TABLE 5

| | | Primer |
|---|---|---|
| Primer | Sequence 5′-3′ | direction |
| 9597 | TGG AAG CAG TAT | Wild type |
| | GTT GGT AAG C | forward |
| | (SEQ ID NO: 29) | |
| oIMR1742 | AAC AGG GAT AGT | Common |
| | AAG ATT GTT CC | reverse |
| | (SEQ ID NO: 30) | |
| olMR1743 | TCC TGT CAT CTC | Mutant |
| | ACC TTG CTC | forward |
| | (SEQ ID NO: 31) | |

The efficacy of using FIX-Tf fusion proteins to treat acute bleeding was evaluated in a tail clip bleeding model following either intravenous (i.v.) injection or oral administration of the above-described fusion proteins.

Efficacy of fusion proteins following i.v. injection. Prior to performing the tail bleed assays, the mice were anesthetized with isoflurane and placed on a heating pad to maintain body temperature. Five minutes following tail vein injections of either 50 IU/kg or 20 IU/kg of the FIX-Tf fusion protein being evaluated, BeneFIX®, or vehicle solution, the distal 4 mm of the tail was clipped. Wild-type mice were used as a control. Blood was collected blood was collected continuously into 13 ml of saline at 37° C. for 15 minutes. Blood loss was determined by quantifying the amount of hemoglobin in the 15-min collection sample. Red blood cells were separated following centrifugation and lysed with hemoglobin reagent (Sigma-Aldrich, St. Louis, Mo., USA). Spectrophometric readings were taken of the lysed samples were read at 540 nm. The total amount of hemoglobin was determined from a standard curve. As shown in FIG. 6A, there was much less blood loss in wild-type mice than in hemophilia B mice (p<0.001).

The 50 IU/kg and 20 IU/kg rFIX-Tf/G₂ treatments in hemophilia B mice significantly reduced blood loss in comparison to the vehicle control in a dose-dependent manner. Thereafter a dose of 20 IU/kg was used for efficacy comparisons. rFIX-Tf/G₂ and rFIX-Tf/SVSQ at 20 IU/kg both significantly reduced blood loss. There were no significant difference between the efficacies of the positive control BeneFIX® and rFIX-Tf/G₂ and rFIX-Tf/SVSQ. The results indicate that rFIX-Tf/G₂ and rFIX-Tf/SVSQ are effective in treating acute bleeding in hemophilia B mice following intravenous injection.

Efficacy of fusion proteins following oral administration. The tail vein bleed model was also used to evaluate the efficacy of the fusion proteins following oral administration by intragastric gavage administration. Efficacy of fusion proteins was tested 18-20 min post administration. The oral efficacy study, showed that 200 IU/kg rFIX-Tf/G₂ treatments in hemophilia B mice significantly reduced blood loss in comparison to the vehicle control, however rFIX-Tf/SVSQ and BeneFIX® at 200 IU/kg had no significant effect for treating acute bleeding (FIG. 6B).

Example 8. Pharmacokinetic study. Pharmacokinetic investigations were performed in wild-type mice following either intravenous (i.v.) injection or oral administration of FIX-Tf fusion proteins described above.

Pharmacokinetics following i.v. injection. Doses of 50 IU/kg of either rFIX-Tf/G₂, rFIX-Tf/SVSQ, or rhFIX (BeneFIX®) were administered by intravenous injection via tail vein injection. A total of 12 time points were used from 2 min to 72 h post administration with 3 animals per points (2 min, 5 min, 10 min, 30 min, 60 min, 90 min, 2 h, 6 h, 12 h, 24 h, 48 h and 72 h). At each time point, animals were anaesthetized with isoflurane and blood collected by heart puncture. Blood samples were stabilized in 0.13 M sodium-citrate (9:1 v/v) and plasma was prepared after centrifugation and stored at −80° C. until analysis. The human FIX specific ELISA was used for determination of FIX concentration at each time point. Pharmacokinetic analyses were performed by nonlinear regression analysis. Half-lives were calculated using the formula t1/2=0.693/k, whereas k is the first-order elimination rate constant obtained from the regression analysis of the terminal phase on a semi-log plot. The area under the curve (AUC) was calculated using the linear trapezoidal method. In vivo recovery was calculated as maximum rise of plasma level (IU/ml)*PV (mL/kg)*100% per dose (IU/kg) where the maximum rise is equal to the maximum concentration obtained from the fitted cure and PV is the assumed plasma volume of 40 mL/kg for mice.

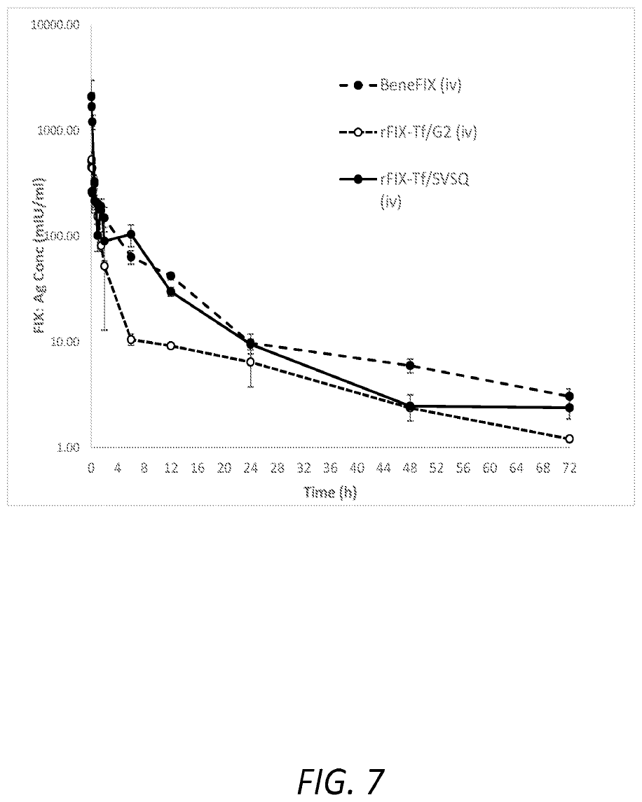

The time courses of plasma levels of rFIX-Tf/G₂, rFIX-Tf/SVSQ and rhFIX (BeneFIX®) following intravenous injection in mice are shown in FIG. 7. rFIX-Tf/G₂, rFIX-Tf/SVSQ and BeneFIX® were cleared by a rapid decline over the first few hours after intravenous injection (Table 6). rFIX-Tf/G₂ and rFIX-Tf/SVSQ were not associated with greater extended half-life than BeneFIX®. The AUC_{fitted} or AUC_{inf} for rhFIX are increased by two-fold in comparison to those values for FIX-Tf/G2. For in-vivo recovery, rFIX-Tf/SVSQ is 4.6 folds higher than that of rFIX-Tf/G2 and wild-type FIX.

TABLE 6

| | Half- | | | In Vivo |
|---|---|---|---|---|
| | life | AUC(mIU/ml*h) | | Recovery |
| Administered protein | (h) | AUC₀₋₇₂ | AUC_{0-inf} | (%) |
| rFIX-Tf/G₂ | 20.44 | 841.27 | 877.01 | 41.41 |
| rFIX-Tf/SVSQ | 24.21 | 1995.15 | 2080.31 | 168.47 |
| BeneFIX ® | 28.82 | 1768.19 | 1895.24 | 36.27 |
| Ratio of rFIX-Tf/G₂ | 0.71 | 0.48 | 0.46 | 1.14 |
| to BeneFIX ® | | | | |
| Ratio of rFIX-Tf/SVSQ | 0.84 | 1.13 | 1.10 | 4.64 |
| to BeneFIX ® | | | | |

Efficacy of fusion proteins following oral administration. The pharmacokinetics following oral administrations were evaluated at a dose of 500 IU/kg. A total of 10 time points were used from 10 min to 72 h post administration with 3 animals per points (10 min, 30 min, 1 h, 2 h, 4 h, 6 h, 12 h, 24 h, 48 h and 72 h). The AUC of rFIX-Tf/G2 calculated from t0 up to the last data point was 1.3 fold higher than wild-type FIX. The AUC values for rFIX-Tf/SVSQ and wild-type FIX were almost the same (FIG. 8 and Table 7).

TABLE 7

| Administered protein | AUC₀₋₇₂ (mIU/ml*h) |
|---|---|
| rFIX-Tf/G₂ | 68.56 |
| rFIX-Tf/SVSQ | 53.83 |
| BeneFIX ® | 52.47 |
| Ratio of rFIX-Tf/G₂ to BeneFIX ® | 1.31 |
| Ratio of rFIX-Tf/SVSQ to BeneFIX ® | 1.03 |

What is claimed:

1. A fusion protein comprising:
A. a coagulation Factor IX (FIX) component;
B. a Transferrin (Tf) component; and
C. a peptide linker,
wherein the peptide linker links the FIX and Tf components.

2. The fusion protein according to claim 1, wherein the peptide linker is cleavable.

3. The fusion protein according to claim 2, wherein the peptide linker is dithiocyclopeptide (SEQ ID NO: 9) or SVSQTSKLTRAETVFPDVDGS (SEQ ID NO: 10).

4. The fusion protein according to claim 1, wherein the peptide linker is non-cleavable.

5. The fusion protein according to claim 4, wherein the peptide linker is: (GGGGS)₂ (SEQ ID NO: 5); (GGGGS)₅ (SEQ ID NO: 6); A(EAAAK)₂A (SEQ ID NO: 7); or A(EAAAK)₅A (SEQ ID NO: 8).

6. The fusion protein according to claim 5, wherein the peptide linker is followed at its carboxyl terminus by the dipeptide, LE.

7. The fusion protein according to claim 1, wherein the peptide linker is the dipeptide, LE.

8. The fusion protein according to claim 1, wherein the FIX component comprises a human-derived FIX amino acid sequence.

9. The fusion protein according to claim 1, wherein the FIX component comprises an amino acid sequence with at least 90% sequence identity to the amino acid sequence of SEQ ID NO: 2, or a fragment thereof.

10. The fusion protein according to claim 1, wherein the Tf component comprises a human-derived Tf amino acid sequence.

11. The fusion protein according to claim 10, wherein the human-derived Tf amino acid sequence comprises an amino acid sequence with at least 90% sequence identity to the amino acid sequence of SEQ ID NO: 4, or a fragment thereof.

12. The fusion protein according to claim 1, wherein the amino acid sequence of the fusion protein comprises an amino acid sequence with at least 90% sequence identity to an amino acid sequence selected from (SEQ ID NO: 12), (SEQ ID NO: 14), (SEQ ID NO: 18), (SEQ ID NO: 20), (SEQ ID NO: 22), or (SEQ ID NO: 24).

13. A polynucleotide, comprising a DNA sequence which encodes a fusion protein according to claim 1.

14. A method for treating a bleeding disorder, comprising administering a fusion protein according to claim 1.

15. The method for treating a bleeding disorder, according to claim 14, wherein the bleeding disorder is Hemophilia B.

16. A method for treating a bleeding disorder according to claim 14, wherein the fusion protein is administered orally.


SEQUENCE LISTING (31 sequences)

SEQ ID NO: 1 - DNA, 1424 bp, Homo sapiens
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactctcgag

SEQ ID NO: 2 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - DNA, 2052 bp, Homo sapiens
ctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 4 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - protein, 10 aa, Artificial
  Linker
GGGGSGGGGS

SEQ ID NO: 6 - protein, 25 aa, Artificial
  Linker
GGGGSGGGGSGGGGSGGGGSGGGGS

SEQ ID NO: 7 - protein, 12 aa, Artificial
  Linker
AEAAAKEAAAKA

SEQ ID NO: 8 - protein, 27 aa, Artificial
  Linker
AEAAAKEAAAKEAAAKEAAAKEAAAKA

SEQ ID NO: 9 - protein, 16 aa, Artificial
  Linker
AGCKNFFPRSFTSCGS

SEQ ID NO: 10 - protein, 21 aa, Artificial
  Linker
SVSQTSKLTRAETVFPDVDGS

SEQ ID NO: 11 - DNA, 3500 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactggaggaggaggatccggaggaggaggatccctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 12 - protein, 1152 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSLEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 13 - DNA, 3533 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactagcgtcagccagactagcaagctgacacgagccgaaaccgtcttccctgatgtcgacggaagcctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 14 - protein, 1163 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTSVSQTSKLTRAETVFPDVDGSLEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 15 - DNA, 3470 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 16 - protein, 1142 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTLEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 17 - DNA, 3551 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactgccgaggccgccgccaaggaggccgccgccaaggaggccgccgccaaggaggccgccgccaaggaggccgccgccaaggccctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 18 - protein, 1169 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTAEAAAKEAAAKEAAAKEAAAKEAAAKALEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 19 - DNA, 3506 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactgccgaggccgccgccaaggaggccgccgccaaggccctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 20 - protein, 1154 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTAEAAAKEAAAKALEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 21 - DNA, 3545 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactggaggaggaggatccggaggaggaggatccggaggaggaggatccggaggaggaggatccggaggaggaggatccctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 22 - protein, 1167 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSLEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 23 - DNA, 3518 bp, Artificial
  Fusion
cttaagaccactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcactgcaggatgtaagaacttcttcccacgtagcttcacaagttgcggtagcctcgaggtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaatctaga

SEQ ID NO: 24 - protein, 1158 aa, Artificial
  Fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTAGCKNFFPRSFTSCGSLEVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 25 - DNA, 32 bp, Artificial
  Primer
accgctcgaggtccctgataaaactgtgagat

SEQ ID NO: 26 - DNA, 31 bp, Artificial
  Primer
gtagtctagattaaggtctacggaaagtgca

SEQ ID NO: 27 - DNA, 32 bp, Artificial; identical to SEQ ID NO: 25
  primer

SEQ ID NO: 28 - DNA, 31 bp, Artificial; identical to SEQ ID NO: 26
  primer

SEQ ID NO: 29 - DNA, 22 bp, Artificial
  primer
tggaagcagtatgttggtaagc

SEQ ID NO: 30 - DNA, 23 bp, Artificial
  primer
aacagggatagtaagattgttcc

SEQ ID NO: 31 - DNA, 21 bp, Artificial
  primer
tcctgtcatctcaccttgctc